(19) United States
(12) Patent Application Publication
(10) Pub. No.: US 2015/0202172 A1
(43) Pub. Date: Jul. 23, 2015

(54) NOVEL METHOD OF USE

(71) Applicant: Glaxo Group Limited, Greenford (GB)

(72) Inventors: Ole GRAFF, Research Triangle Park, NC (US); Susan Marie LEARNED, Research Triangle Park, NC (US); Virginia SCHMITH, Research Triangle Park, NC (US); Rajendra SINGH, King of Prussia, PA (US)

(73) Assignee: Glaxo Group Limited, Greenford (GB)

(21) Appl. No.: 14/273,808

(22) Filed: May 9, 2014

Related U.S. Application Data
(60) Provisional application No. 61/930,201, filed on Jan. 22, 2014.

(51) Int. Cl. A61K 31/203

(52) U.S. Cl. CPC A61K 31/203

(57) ABSTRACT

The present invention is directed to methods of treating elderly patients with Alitretinoin severe chronic hand eczema (CHE), refractory to treatment with potent topical corticosteroids. One embodiment of the invention is a method of improving the efficacy of Alitretinoin treatment for Severe Refractory Chronic Hand Eczema (CHE) comprising
a) identifying and/or selecting a patient in need of treatment, wherein the age of the patient is 50 years old or greater;
b) administering an effective amount of Alitretinoin to said patient;
c) determining PGA score; and
d) discontinuing treatment with Alitretinoin when said patient achieves the PGA score of 1 or 2; thereby. The improvement is also expected to reduce risks in the patient population as continuing treatment will not be necessary.

### FIELD OF THE INVENTION

The present invention is directed to improved efficacy for use of Alitretinoin in an elderly patient population with Severe Refractory Chronic Hand Eczema (CHE) and to show that treatment should be discontinued once patients have responded to Alitretinoin.

### BACKGROUND OF THE INVENTION

Alitretinoin (9-cis isomer of retinoic acid (RA), Toctino®) is a physiological metabolite of vitamin A (retinol) and belongs to the chemical class of retinoids comprising natural as well as synthetic compounds. Alitretinoin is an investigational product in the United States (US) and an approved product in multiple European countries, Canada, and Israel for the treatment of severe chronic hand eczema (CHE), refractory to treatment with potent topical corticosteroids. Hands are highly visible and are critical for activities of daily living in both the workplace and home, and used for communication and expression [Bissonette, et al., JEADV; 24 (Suppl. S3): 1-20, (2010)]. Alitretinoin for use in the treatment of chronic hand eczema is disclosed in, Bollag et al., U.S. Pat. No. 6,589,989, and for eczema in Bollag et al., U.S. Pat. No. 8,541,469.

Severe CHE strongly affects physical function, work and productivity (physical role), body pain, general health, vitality, social function, emotional and mental health, with negative scores in all dimensions of the Short Form Health Survey (SF-36) [Bingefors, et al., Acta Derm Venereol; 91: 452-458, (2011); Boehm, et al., Contact Dermatitis; 67: 184-192, (2012)]. This condition can be so severe that the impact on health-related quality of life of patients suffering from severe CHE was rated greater than the experiences of patients with asthma or psoriasis [Moberg, et al., Br J Dermatol, 161:12: 397-403, (2009)]. Severe CHE is associated with potentially disabling functional impairment and persistent and disfiguring changes in hand appearance and function [Coenraads, et al., Br J Dermatol, 152;2: 296-301, (2005)] and is characterized by thick scaly skin with painful fissures, itching, vesicles and excoriation, erythema, and edema. Pain, itching, and bleeding from fissures can make manual work (e.g., buttoning a shirt, opening a door) difficult to perform, and the embarrassment caused by the appearance of the skin and nails may prevent participation in social or work employment situations [English, et al., Clin Dermatol, 29: 273-277, (2011)].

There are no treatments approved for severe refractory CHE in the Unites States. Because of the significant safety issues associated with current treatments that are used, and because the evidence demonstrating the safe and effective use of these treatments is very limited [Van Coevorden, et al., Arch Dermatol, 140:12: 1463-1466, (2004)], patients with severe CHE refractory to topical corticosteroids have no consistently reliable and safe therapeutic options for their chronic condition.

Many clinical trials have been done with Alitretinoin capsules for the treatment of severe refractory CHE but optimal benefit to risk ratio in subjects with severe refractory CHE has not been defined. The well-established risks associated with oral retinoids imply that the treatment should be limited to only those subjects with the potential to significantly benefit and that treatment should be limited to the minimum time needed for adequate response, instead of chronic therapy for an undefined duration. Therefore, long term benefit in this situation is best defined as the time at which patients can no longer be controlled with topical corticosteroids following Alitretinoin treatment. This time would be when the subject relapses to severe disease (such as by a PGA score of 5) and becomes refractory to, or intolerant of, topical corticosteroids.

The present invention is directed to an improved benefit in elderly patients when taking alitretinoin for severe refractory CHE and to improved benefit:risk ratio in all patients receiving alitretinoin by selecting the optimal duration.

### SUMMARY OF THE INVENTION

The present invention is directed to a method of improving the efficacy of Alitretinoin for the treatment of Severe Refractory Chronic Hand Eczema (CHE) in an elderly patient population in need thereof comprising administering to said elderly patient an effective amount of Alitretinoin. Suitably the age of the patient is 50 years of age or older. In another embodiment, the patient is 55 years of age or greater. In another embodiment, the patient is 60 years of age or greater. In another embodiment, the patient is 65 years of age or greater. In another embodiment, the patient is 70 years of age or greater. In another embodiment, the patient is 75 years of age or greater.

Suitably, the disease severity of the CHE may be evaluated using a modified total lesion symptom score (mTLSS) and/or a PGA scale. Suitably the Alitretinoin is administered until the patient reaches a PGA score of clear or almost clear and wherein the time to relapse of the patient is 6 months or greater. Suitably, if the patient achieves a PGA score of 1 or 2 during the course of treatment with Alitretinoin the course of treatment is discontinued.

Suitably the amount of Alitretinoin administered is from about 10 mg to about 40 mg per day. In one embodiment the Alitretinoin administered is about 10 mg or about 30 mg. In one embodiment the Alitretinoin administered is about 30 mg.

Suitably, a course of treatment with Alitretinoin is a 24 week of treatment. In one embodiment, the course of treatment is less than the 24 week duration. In another embodiment the Alitretinoin administered for about 8 weeks or less. In another embodiment, the Alitretinoin administered for about 12 weeks or less. In another embodiment, the Alitretinoin administered for about 16 weeks or less. In another embodiment, the Alitretinoin administered for about 20 weeks or less.

In one embodiment where the patient population is 50 years or greater, the patient experiences a faster onset of action, and/or a higher response rate to the treatment with Alitretinoin, and/or a greater chance of not relapsing 6 months after treatment. In another embodiment, the time to relapse of this patient population post treatment is 6 months or greater. In another embodiment the time to patient relapse is not influenced by the duration of treatment with Alitretinoin.

Another embodiment of the invention is a method of obtaining optimal dosing of Alitretinoin for the treatment of Severe Refractory Chronic Hand Eczema (CHE) in a patient in need thereof comprising administering to said patient an effective amount of Alitretinoin, the age of the patient is 50 years old or greater, and the patient is treated until a PGA score of clear or almost clear is reached.

In one embodiment, the patient is 55 years of age or greater. In another embodiment, the patient is 60 years of age or greater. In another embodiment, the patient is 65 years of age or greater. In another embodiment, the patient is 70 years of age or greater. In another embodiment, the patient is 75 years of age or greater.

In one embodiment, there are less patients that relapse 6 months post-treatment in the 50 year of age or greater population as compared to placebo or compared to a population of less than 50 years of age. In one embodiment the time to relapse of the patient is 6 months or greater in >83% of subjects.

In one embodiment the treatment time to obtain a PGA score of clear or almost clear is less than the duration of treatment as compared to placebo or to a population less than 50 years of age. In one embodiment the patient population of 50 years or greater is less likely to incur an increased risk from treatment with Alitretinoin as compared to placebo or to a population less than 50 years of age. In another embodiment, the elderly patient population experiences a faster onset of action and/or a higher response rate to the treatment with Alitretinoin than a patient population younger than 50 years of age. In another embodiment the time to relapse is not influenced by the duration of treatment.

In one embodiment the time to relapse of the patient, post treatment is 6 months or greater. In one embodiment the time to relapse of the patient, post treatment is 9 months or greater.

Suitably, a course of treatment with Alitretinoin is a 24 week of treatment. In one embodiment, the course of treatment is less than the 24 week duration. In another embodiment the Alitretinoin administered for about 8 weeks or less. In another embodiment, the Alitretinoin administered for about 12 weeks or less. In another embodiment, the Alitretinoin administered for about 16 weeks or less. In another embodiment, the Alitretinoin administered for about 20 weeks or less.

Suitably the amount of Alitretinoin administered is from about 10 mg to about 40 mg per day. In one embodiment the Alitretinoin administered is about 10 mg or about 30 mg. In one embodiment the Alitretinoin administered is about 30 mg.

In one embodiment, there are less patients that relapse 6 months post-treatment in the 50 year of age or greater population as compared to placebo or compared to a population of less than 50 years of age.

Another embodiment of the invention is a method of decreasing the time to onset and the duration of treatment time in a patient with Severe Refractory Chronic Hand Eczema (CHE) comprises administering to said patient an effective amount of Alitretinoin, and wherein the age of the patient is 50 years old or greater.

Another embodiment of the invention is a method of improving the likelihood of having an adverse event in an elderly patient having Severe Refractory Chronic Hand Eczema (CHE) comprising administering to said patient an effective amount of Alitretinoin, and discontinuing treatment when said patient achieves a PGA score or 1 or 2, and wherein the age of the patient is 50 years old or greater.

Another aspect of the invention is a method of improving benefit:risk in a patient taking Alitretinoin for severe refractory CHE who responds to Alitretinoin treatment with a PGA score of 1 (clear) or 2 (almost clear) hands by shortening the treatment duration as compared to placebo.

Another aspect of the invention is a method of improving benefit:risk in a patient taking Alitretinoin for severe refractory CHE who responds to Alitretinoin treatment with a PGA score of 1 (clear) or 2 (almost clear) hands by shortening the treatment duration from a 24 week duration, thereby reducing the risk of adverse events from continuing treatment.

Another aspect of the invention is the identification of the optimal duration of dosing in a patient when taking Alitretinoin for severe refractory CHE
a) by identifying and/or selecting a patient in need of treatment,
b) administering Alitretinoin to said patient
c) continuing treatment until the patient has reached a PGA score of 1 (clear) or 2 (almost clear hands);
d) discontinuing treatment, and monitoring for relapse of CHE, wherein the rate of relapse is reduced over placebo or a patient who did not achieve a PGA score of 1 or 2.

Another aspect of the invention is a method of improving the safety profile of Alitretinoin administered for the treatment for Severe Refractory Chronic Hand Eczema (CHE) comprising
a) identifying and/or selecting a patient in need of treatment,
b) administering an effective amount of Alitretinoin to said patient;
c) determining PGA score;
d) discontinuing treatment with Alitretinoin when said patient achieves the PGA score of 1 or 2; and
e) wherein the duration of treatment is shortened as compared to placebo.

In a further embodiment, suitably the age of the patient is 50 years old or greater, alternatively 60 years old or greater, or 65 years of age or greater, 70 years old or greater, or 75 years of age or greater.

In another embodiment of the invention there is a method of improving the safety profile of Alitretinoin treatment for Severe Refractory Chronic Hand Eczema (CHE) comprising
a) identifying and/or selecting a patient in need of treatment, wherein the age of the patient is 50 years old or greater;
b) administering an effective amount of Alitretinoin to said patient;
c) determining their PGA score; and
d) discontinuing treatment with Alitretinoin when said patient achieves the PGA score of 1 or 2; and
e) wherein the likelihood of relapse post 6 months treatment is less for a patient of 50 years old or greater when compared to placebo or to a patient of <50 years of age.

Another aspect of the invention is a method of improving the likelihood of having an adverse event in an elderly patient having Severe Refractory Chronic Hand Eczema (CHE) comprising administering to said patient an effective amount of Alitretinoin, and discontinuing treatment when said patient achieves a PGA score or 1 or 2, and wherein the age of the patient is 50 years old or greater.

Another embodiment of the invention is a method of improving the efficacy of Alitretinoin treatment for Severe Refractory Chronic Hand Eczema (CHE) comprising
a) identifying and/or selecting a patient in need of treatment;
b) administering an effective amount of Alitretinoin to said patient;
c) determining the patients PGA score; and
d) discontinuing treatment with Alitretinoin when said patient achieves the PGA score of 1 or 2; or alternatively a drop in the patients PGA score by >2.

Another embodiment of the invention is a method of improving the efficacy of Alitretinoin treatment for Severe Refractory Chronic Hand Eczema (CHE) comprising
1) identifying and/or selecting a patient in need of treatment, wherein the age of the patient is 50 years old or greater;
2) administering an effective amount of Alitretinoin to said patient;
3) determining PGA score; and
4) discontinuing treatment with Alitretinoin when said patient achieves the PGA score of 1 or 2.

Another embodiment of the invention is a method of improving the time to response for patients taking Alitretinoin for Severe Refractory Chronic Hand Eczema (CHE) comprising
1) identifying and/or selecting a patient in need of treatment, wherein the age of the patient is 50 years old or greater;
2) administering an effective amount of Alitretinoin to said patient;
3) determining PGA score; and
4) discontinuing treatment with Alitretinoin when said patient achieves a drop of 2 or more points in the PGA scale or achieve a PGA score of 1 or 2; and
5) when the time to response for said patient is treatment is shortened as compared to placebo or a patient of <50 years of age.

Another aspect of the invention is Alitretinoin for use in the treatment of Severe Refractory Chronic Hand Eczema (CHE) according to each of the methods described above and further herein.

Another aspect of the invention is the use of Alitretinoin in the manufacture of a medicament for use in the treatment of Severe Refractory Chronic Hand Eczema (CHE) according to each of the methods described above, and further herein.

### DETAILED DESCRIPTION OF THE INVENTION

The present invention is directed to improving the treatment of patients having severe refractory chronic hand eczema (CHE) regardless of their age. It has surprisingly been discovered that there is no relationship between the duration of treatment and the likelihood of relapse in those patients who respond to Alitretinoin treatment (i.e., a longer duration of treatment does not lead to improved lack of relapse). This data means that Alitretinoin treatment can be discontinued once a patient achieves clear or almost clear hands, and there is no need to continue treating these patients for a full 24 week treatment course. It has surprisingly been discovered that age significantly influences the time course of efficacy of Alitretinoin for CHE. The elderly show an earlier response to treatment with Alitretinoin. The elderly also show a more pronounced response to treatment with Alitretinoin. The elderly patient may thus be more likely to respond earlier, and require a shorter treatment duration, and be more unlikely to incur an increased risk of adverse events associated with longer treatment durations.

A full course of treatment, as defined herein, is administration once daily of Alitretinoin for 24 weeks. In another embodiment the course of treatment is less than the 24 week duration of treatment. In another embodiment the Alitretinoin administered for about 8 weeks or less. In another embodiment, the Alitretinoin administered for about 12 weeks or less. In another embodiment, the Alitretinoin administered for about 16 weeks or less. In another embodiment, the Alitretinoin administered for about 20 weeks or less.

In an alternative embodiment, Alitretinoin is administered for up to 20 weeks. In another embodiment Alitretinoin is administered for up to 16 weeks. In another embodiment Alitretinoin is administered for up to 12 weeks. In another embodiment, Alitretinoin is administered for 8 weeks. In yet another embodiment, Alitretinoin is administered for 4 weeks.

Elderly as defined herein is a patient of 50 years of age or greater. In one embodiment the elderly patient is 55 years of age or greater. In one embodiment the elderly patient is 60 years of age or greater. In one embodiment the elderly patient is 65 years of age or greater. In another embodiment the elderly patient is 70 years of age or greater. In one embodiment the elderly patient is 75 years of age or greater.

Alitretinoin may be dosed once daily from 10mg to 40mg for CHE. In one embodiment the daily dose is from 10 to 30mg. In one embodiment the daily dosage is 10 mg. In another embodiment the daily dosage is 20mg. In another embodiment the daily dosage is 30 mg. In another embodiment the daily dosage is 40mg. Suitably, the initial daily dosage for a patient, including elderly patients, is 30mg.

It has also been found that in subjects who achieve a Physicians Global Assessment (PGA) score of 1 (clear) or 2 (almost clear) there is no relationship between the duration of treatment and the relapse status at 6 months post treatment. Therefore, once the patient has responded there is no added benefit to the patient to continue treatment. One patient population of particular interest is the elderly population. While it is also recognized that the entire population of patients taking Alitretinoin for CHE will benefit from this, the elderly patient population also experiences a faster onset of action and an improved response to efficacy over the general population. The currently prescribed recommendation for treatment is 12 to 24 weeks, thus optimally; knowing that achieving a “clear” or “almost clear” hands score will allow the patient will discontinue treatment earlier.

When the findings in elderly were discovered, the proportion of overall responders (PGA score of 1+2) at the end of treatment in patients 65 or 75 years of age was found to be higher in both the placebo and treatment groups, with magnitude of efficacy, relative to placebo, of 22%, 28%, and 29% in a 30 year old, 65 year old, and 75 year old respectively (i.e., a difference of 7% between a 30 and 75 year old). In addition, advanced age was associated with faster time to onset of effect (>2 weeks faster in a 65 or a 75 year old than in a 30 year old) and a higher probability of not relapsing (i.e., not having a PGA score=5) 6 months post treatment (87%, 89% and 79% in a 65, 75 or 30 year old, respectively).

The data indicates a higher efficacy at end of treatment in the elderly is driven by those subjects who have a PGA score of 1 (Table 2).

For those patients who achieve a PGA=1 at end of treatment; the overall comparison of efficacy, relative to placebo, between 30 and 75 year old is 8%.

For those patients who achieve a PGA=2 at end of treatment, there were no differences in magnitude of efficacy between 30 year old (13.5%) and 75 year old (12.8%).

For those patients who achieve a PGA=3 at end of treatment (also defined as a partial responder), the overall comparison of a PGA score of 3 at end of treatment is 8% greater for young patients receiving Alitretinoin, relative to placebo, but similar between young and elderly patients. A PGA score of 3 is considered a partial response to treatment.

In addition to the effect of age on the magnitude of efficacy at the end of treatment, there are additional advantages in elderly patients post treatment, whether evaluated as a PGA score of 1 and 2 combined or as a PGA score of 1, 2, or 3 evaluated independently.

The proportion of overall responders, e.g. those having a PGA score of PGA=1 or PGA=2, (all individually) at the end of treatment show a 10% difference between a 75 year old and 30 year old in the proportion of patients who did not relapse (i.e., PGA score≠5) 6 months after treatment. Thus, the elderly are i) less likely to relapse than a younger subject and ii) the likelihood of relapse is not related to whether you have a PGA score of 1 or 2. Relative to placebo, Alitretinoin 30 mg administered for 8 to 24 weeks results in approximately 4% to 7% of new responders (PGA score of 1 or 2) occurring each additional 4 weeks of treatment after the initial 8 week treatment period.

If the goal is a PGA score of=1 at end of treatment, the response rate increases by 2-3% every month, relative to placebo, between 8 and 24 weeks.

If the goal is a PGA score of=2 at end of treatment, the response rate increases by 2-3% every month, relative to placebo between 8 and 24 weeks.

If the goal is a PGA score of=3 at end of treatment, the partial response rate (PGA=3) remains constant, relative to placebo, regardless of treatment duration.

Thus, increasing response rates are seen with increasing treatment duration for both a PGA score of 1 and 2, whether analyzed as combined or as individual events, as expected. Some patients may respond quickly (e.g., after 8 weeks), while others may take 12 to 24 weeks to respond. The surprising finding is that there is no relationship between duration of treatment and relapse 6 months post treatment. That is, shortening treatment did not necessarily result in a higher chance of relapse at 6 months post treatment. Specifically, in those patients with a PGA score=1 or 2, >83% of subjects who responded to Alitretinoin would not return to severe disease within 6 months post treatment (Table 4).

When the effect of treatment duration on relapse at 6 months post-treatment is looked at in those with a PGA=1 or 2 after 12 to 24 weeks of treatment, 82% to 84% of subjects who responded to Alitretinoin do not relapse 6 months post treatment, regardless of treatment duration (Table 4).

More specifically analysis showed that for those with a PGA score of=1 after 12 to 24 weeks of treatment, 82% to 84% of subjects who responded to Alitretinoin do not relapse 6 months post treatment, regardless of treatment duration (Table 6).

For those with a PGA score of=2 after 12 to 24 weeks of treatment, 81% to 83% of subjects who responded to Alitretinoin do not relapse 6 months post treatment, regardless of treatment duration (Table 6).

For those with a PGA score of=3 after 12 to 24 weeks of treatment, 79% to 82% of subjects who responded to Alitretinoin do not relapse 6 months post treatment, regardless of treatment duration (Table 6).

Thus, one can conclude that there is no relationship between treatment duration and lack of relapse. This establishes that there is no added benefit to continuing treatment once the patient has responded. While the optimal duration of treatment still appears to be between 12 and 24 weeks, some patients may respond as early as 4-8 weeks, and one should therefore discontinue treatment upon reaching a PGA score of “clear” or “almost clear” hands. One embodiment of the invention is a method of treatment of a patient having CHE with an effective amount of Alitretinoin, once daily for an 8 week or less duration of time. Another embodiment of the invention is a method of treatment of a patient having CHE with an effective amount of Alitretinoin, once daily for an 4 week or less duration of time.

Another embodiment of the invention is a method of improving the efficacy of Alitretinoin treatment for Severe Refractory Chronic Hand Eczema (CHE) comprising
b) identifying and/or selecting a patient in need of treatment;
b) administering an effective amount of Alitretinoin to said patient;
c) determining the patients PGA score; and
d) discontinuing treatment with Alitretinoin when said patient achieves the PGA score of 1 or 2; or alternatively a drop in the patients PGA score by >2.

In one embodiment the age of the patient is 50 years of age or greater. In another embodiment the patient is 55 years of age or greater. In another embodiment the patient is 60 years old or greater. In another embodiment the age of the patient is 65 years old or greater. In another embodiment the age of the patient is 70 years old or greater. In one embodiment the age of the patient is 75 years old or greater. In one embodiment the likelihood of relapse post 6 months treatment is less for a patient of 50 years old or greater when compared to placebo.

In another embodiment the patient experiences a faster onset of action and/or a higher response rate to the treatment with Alitretinoin.

In another embodiment the duration of treatment is decreased as compared to placebo. Suitably the Alitretinoin administered is for up to 24 weeks. In another embodiment the course of treatment is less than the 24 week duration of treatment. In another embodiment the Alitretinoin administered for about 8 weeks or less. In another embodiment, the Alitretinoin administered for about 12 weeks or less. In another embodiment, the Alitretinoin administered for about 16 weeks or less. In another embodiment, the Alitretinoin administered for about 20 weeks or less.

Suitably the amount of Alitretinoin administered is from about 10 mg to about 40mg per day. In one embodiment the Alitretinoin administered is about 10 mg or about 30 mg. In one embodiment the Alitretinoin administered is about 30 mg.

In another embodiment the time to relapse post treatment is 6 months or greater. In another embodiment the time to relapse post treatment is 9 months or greater.

Another embodiment of the invention is a method of improving the efficacy of Alitretinoin treatment for Severe Refractory Chronic Hand Eczema (CHE) comprising
5) identifying and/or selecting a patient in need of treatment, wherein the age of the patient is 50 years old or greater;
6) administering an effective amount of Alitretinoin to said patient;
7) determining PGA score; and
8) discontinuing treatment with Alitretinoin when said patient achieves the PGA score of 1 or 2.

In one embodiment the age of the patient is 60 years old or greater. In one embodiment the age of the patient is 65 years old or greater. In another embodiment the age of the patient is 70 years old or greater. In one embodiment the age of the patient is 75 years old or greater. In another embodiment the patient experiences a faster onset of action and/or a higher response rate to the treatment with Alitretinoin.

In another embodiment the duration of treatment is shortened as compared to a patient of <50 years of age. In another embodiment, the proportion of those patients who do not relapse 6 months post treatment is greater in those subjects that are >65 years of age, or >70 years of age, or >75 years of age. In another embodiment the time to relapse post treatment is 9 months or greater. In another embodiment the time to relapse post treatment is 6 months or greater.

Suitably the Alitretinoin administered is for up to 24 weeks. In another embodiment the course of treatment is less than the 24 week duration of treatment. In another embodiment the Alitretinoin administered for about 8 weeks or less. In another embodiment, the Alitretinoin administered for about 12 weeks or less. In another embodiment, the Alitretinoin administered for about 16 weeks or less. In another embodiment, the Alitretinoin administered for about 20 weeks or less.

Another aspect of the invention is a method of improving the safety profile of Alitretinoin administered for the treatment for Severe Refractory Chronic Hand Eczema (CHE) comprising
a) identifying and or selecting a patient in need of treatment, wherein the age of the patient is 50 years old or greater;
b) administering an effective amount of Alitretinoin to said patient;
c) determining PGA score;
d) discontinuing treatment with Alitretinoin when said patient achieves the PGA score of 1 or 2;
e) wherein the duration of treatment is shortened as compared to placebo; and/or or a patient of <50 years of age; and.
f) wherein the rate of relapse is prolonged if said patient achieves a PGA score of 1 or 2 at end of treatment.

In one embodiment the age of the patient is 55 years old or greater. In one embodiment the age of the patient is 60 years old or greater. In one embodiment the age of the patient is 65 years old or greater. In another embodiment the age of the patient is 70 years old or greater. In one embodiment the age of the patient is 75 years old or greater.

In another embodiment the patient experiences a faster onset of action and/or a higher response rate to the treatment with Alitretinoin. By achieving a PGA score of 1 or 2 at end of treatment, the patient has an increased chance of not relapsing within a ≧6 month time period, thus reducing the need for another treatment round of Alitretinoin.

In another embodiment the duration of treatment is shortened as compared to placebo. In another embodiment the duration of treatment is shortened as compared to a patient of <50 years of age. In another embodiment the time to relapse post treatment is 6 months or greater. In another embodiment the time to relapse post treatment is 9 months or greater.

Suitably the amount of Alitretinoin administered is from about 10 mg to about 40 mg per day. In one embodiment the Alitretinoin administered is about 10 mg or about 30 mg. In one embodiment the Alitretinoin administered is about 30 mg.

Another aspect of the invention is a method of improving the time to response for patients taking Alitretinoin for Severe Refractory Chronic Hand Eczema (CHE) comprising
1) identifying and/selecting a patient in need of treatment, wherein the age of the patient is 50 years old or greater;
2) administering an effective amount of Alitretinoin to said patient;
3) determining PGA score; and
4) discontinuing treatment with Alitretinoin when said patient achieves a drop of 2 or more points in the PGA scale or achieve a PGA score of 1 or 2.

In one embodiment, the age of the patient is 60 years old or greater. In one embodiment, the age of the patient is 65 years old or greater. In another embodiment, the age of the patient is 70 years old or greater. In one embodiment, the age of the patient is 75 years old or greater.

In another embodiment, the patient experiences a faster onset of action and/or a higher response rate to the treatment with Alitretinoin.

In another embodiment, the duration of treatment is shortened as compared to placebo and/or a patient of <50 years of age. In one embodiment, the time to relapse post treatment is 6 months or greater, or the time to relapse post treatment is 9 months or greater.

In another embodiment, the patient population of 50 years or greater is less likely to incur an increased risk from treatment with Alitretinoin as compared to placebo or to a population less than 50 years of age.

Suitably, the amount of Alitretinoin administered is from about 10 mg to about 40 mg per day. In one embodiment the Alitretinoin administered is about 10 mg or about 30 mg. In one embodiment the Alitretinoin administered is about 30 mg.

Another aspect of the invention is a method of improving the safety profile of Alitretinoin treatment for Severe Refractory Chronic Hand Eczema (CHE) comprising
a) identifying and/or selecting a patient in need of treatment, wherein the age of the patient is 50 years old or greater;
b) administering an effective amount of Alitretinoin to said patient;
c) determining their PGA score; and
d) discontinuing treatment with Alitretinoin when said patient achieves the PGA score of 1 or 2; and
e) wherein the likelihood of relapse post 6 months treatment is less for a patient of 50 years old or greater.

In one embodiment, the age of the patient is 55 years old or greater. In one embodiment, the age of the patient is 60 years old or greater. In one embodiment, the age of the patient is 65 years old or greater. In another embodiment, the age of the patient is 70 years old or greater. In one embodiment the age of the patient is 75 years old or greater.

In another embodiment, the patient experiences a faster onset of action and/or a higher response rate to the treatment with Alitretinoin.

In another embodiment, the duration of treatment is shortened as compared to placebo or a patient of <50 years of age. In another embodiment, the likelihood of relapse post 6 months treatment is less when compared to placebo.

In one embodiment the time to relapse of the patient, post treatment is 6 months or greater. In one embodiment the time to relapse of the patient, post treatment is 9 months or greater.

Suitably the Alitretinoin administered is for up to 24 weeks. In another embodiment, the course of treatment is less than the 24 week duration of treatment. In another embodiment, the Alitretinoin administered for about 8 weeks or less. In another embodiment, the Alitretinoin administered for about 12 weeks or less. In another embodiment, the Alitretinoin administered for about 16 weeks or less. In another embodiment, the Alitretinoin administered for about 20 weeks or less.

Suitably, the amount of Alitretinoin administered is from about 10 mg to about 40 mg per day. In one embodiment the Alitretinoin administered is about 10 mg or about 30 mg. In one embodiment the Alitretinoin administered is about 30 mg.

Another embodiment of the invention is a method of decreasing the time to onset and the duration of treatment time in a patient with Severe Refractory Chronic Hand Eczema (CHE) comprises administering to said patient an effective amount of Alitretinoin, and wherein the age of the patient is 50 years old or greater.

In one embodiment the patient determines their PGA score; and discontinues treatment with Alitretinoin when the patient achieves the PGA score of 1 or 2. In one embodiment the time to relapse post treatment of the patient is 6 months or greater.

In one embodiment the age of the patient is 55 years old or greater. In one embodiment the age of the patient is 60 years old or greater. In one embodiment the age of the patient is 65 years old or greater. In another embodiment the age of the patient is 70 years old or greater. In one embodiment the age of the patient is 75 years old or greater.

In another embodiment the patient experiences a faster onset of action and/or a higher response rate to the treatment with Alitretinoin.

In another embodiment the duration of treatment is shortened as compared to placebo or a patient of <50 years of age. In another embodiment the likelihood of relapse post 6 months treatment is less when compared to placebo.

In one embodiment the time to relapse of the patient, post treatment is 6 months or greater. In one embodiment the time to relapse of the patient, post treatment is 9 months or greater.

Suitably the course of treatment is less than a 24 week duration of treatment. In another embodiment the Alitretinoin administered for about 8 weeks or less. In another embodiment, the Alitretinoin administered for about 12 weeks or less. In another embodiment, the Alitretinoin administered for about 16 weeks or less. In another embodiment, the Alitretinoin administered for about 20 weeks or less.

Suitably the amount of Alitretinoin administered is from about 10 mg to about 40 mg per day. In one embodiment the Alitretinoin administered is about 10 mg or about 30 mg. In one embodiment the Alitretinoin administered is about 30 mg.

Another aspect of the invention is a method of improving the likelihood of having an adverse event in an elderly patient having Severe Refractory Chronic Hand Eczema (CHE) comprising administering to said patient an effective amount of Alitretinoin, and discontinuing treatment when said patient achieves a PGA score or 1 or 2, and wherein the age of the patient is 50 years old or greater.

In one embodiment the age of the patient is 55 years old or greater. In one embodiment the age of the patient is 60 years old or greater. In one embodiment the age of the patient is 65 years old or greater. In another embodiment the age of the patient is 70 years old or greater. In one embodiment the age of the patient is 75 years old or greater.

In another embodiment the patient experiences a faster onset of action and/or a higher response rate to the treatment with Alitretinoin.

In another embodiment the duration of treatment is shortened as compared to placebo or a patient of <50 years of age. In another embodiment the likelihood of relapse post 6 months treatment is less when compared to placebo.

In one embodiment the time to relapse of the patient, post treatment is 6 months or greater. In one embodiment the time to relapse of the patient, post treatment is 9 months or greater.

Suitably the course of treatment is less than a 24 week duration of treatment. In another embodiment the Alitretinoin administered for about 8 weeks or less. In another embodiment, the Alitretinoin administered for about 12 weeks or less. In another embodiment, the Alitretinoin administered for about 16 weeks or less. In another embodiment, the Alitretinoin administered for about 20 weeks or less.

Suitably the amount of Alitretinoin administered is from about 10 mg to about 40 mg per day. In one embodiment the Alitretinoin administered is about 10 mg or about 30 mg. In one embodiment the Alitretinoin administered is about 30 mg.

Another aspect of the invention is Alitretinoin for use in the treatment of Severe Refractory Chronic Hand Eczema (CHE) according to each of the methods described above. Further provided is Alitretinoin for use in the treatment of Severe Refractory Chronic Hand Eczema (CHE) according to each specific embodiment associated with each method described above.

Another aspect of the invention is the use of Alitretinoin in the manufacture of a medicament for use in the treatment of Severe Refractory Chronic Hand Eczema (CHE) according to each of the methods described above. Further provided is the use of Alitretinoin in the manufacture of a medicament for use in the treatment of Severe Refractory Chronic Hand Eczema (CHE) according to each specific embodiment associated with each method described above.

Suitable formulations for Alitretinoin for use herein are described in WO 2005/048994 (US 2010/0136108) which is incorporated herein by reference in its entirety.

While the safety profile and adverse effects of Alitretinoin have been well documented, a suitable review paper is Schmitt-Hoffmann, et al, Clinical and Experimental Dermatology, British Association of Dermatologists, 36 (Suppl. 2), 29-34 29, (2011).

As can be seen in the tables below, Table 1 demonstrates the relationship between age and the time to onset of effect, proportion of responders at end of treatment and proportion of responders who do not relapse 6 months after treatment cessation.

TABLE 1

| | | Time to Onset | | Proportion of |
|---|---|---|---|---|
| | | of Effect | Proportion of | Responders Who |
| | | (a Drop of 2 | Responders | Did Not Relapse |
| | | or More Points | (PGA = 1 or 2) | (PGA ≠ 5) |
| | | in PGA in | at End | 6 months |
| | | 30% of Subjects) | of Treatment | Post Treatment |
| Age | Treatment | [Days] | [%] | [%] |
| 30 yr | Placebo | 88 (63, 140) | 11.8 (7, 18) | 71.5 (50, 90) |
| | 30 mg | 58 (49, 70) | 33.9 (26, 42) | 78.7 (67, 90) |
| 50 yr | Placebo | 63 (49, 84) | 16.0 (10, 22) | 77.0 (60, 93) |
| | 30 mg | 49 (42, 56) | 41.1 (33, 49) | 83.2 (73, 92) |
| 65 yr | Placebo | 51 (42, 63) | 20.3 (14, 27) | 81.4 (67, 94) |
| | 30 mg | 43 (35, 49) | 48.0 (40, 56) | 86.6 (78, 94) |
| 75 yr | Placebo | 46 (35, 56) | 24.0 (17, 31) | 84.1 (72, 96) |
| | 30 mg | 39 (35, 42) | 53.0 (45, 62) | 88.7 (81, 95) |

Table 2 demonstrates in greater detail of the relationship between age and a specific PGA score of 1, 2 or 3 (evaluated individually) at end of treatment:

TABLE 2

| | | Proportion | Proportion | Proportion |
|---|---|---|---|---|
| | | of Subjects | of Subjects | of Subjects |
| | | (PGA = 1) | (PGA = 2) | (PGA = 3) |
| | | at End of | at End of | at End of |
| | | Treatment | Treatment | Treatment |
| Age | Treatment | [%] | [%] | [%] |
| 30 yr | Placebo | 2.7 (1, 5) | 9.4 (5, 14) | 20.2 (14, 27) |
| | 30 mg | 10.9 (6, 17) | 22.9 (16, 30) | 28.4 (21, 36) |
| 50 yr | Placebo | 3.8 (1, 7) | 12.4 (7, 18) | 23.2 (17, 30) |
| | 30 mg | 14.9 (9, 21) | 26.3 (20, 34) | 28.3 (20, 35) |
| 65 yr | Placebo | 5.3 (2, 9) | 15 (9, 21) | 25.8 (19, 33) |
| | 30 mg | 19.3 (13, 26) | 28.7 (22, 36) | 27.1 (19, 35) |
| 75 yr | Placebo | 6.7 (3, 11) | 17.4 (11, 24) | 27.1 (19, 34) |
| | 30 mg | 22.8 (16, 30) | 30.2 (23, 37) | 25.8 (18, 33) |

Table 3 demonstrates in greater detail of the relationship between age, a specific PGA score of 1, 2 or 3 (evaluated individually) at end of treatment, and the likelihood of not relapsing (PGA score ≠5) 6 months after cessation of dosing with alitretinoin:

TABLE 3

| | | Proportion | Proportion | Proportion |
|---|---|---|---|---|
| | | of Subjects | of Subjects | of Subjects |
| | | with PGA = 1 | with PGA = 2 | with PGA = 3 |
| | | at end of | at the end of | at the end of |
| | | treatment who | treatment who | treatment who |
| | | did not relapse | did not relapse | did not relapse |
| | | (PGA ≠ 5) | (PGA ≠ 5) | (PGA ≠ 5) |
| | | 6 Months Post | 6 Months Post | 6 Months Post |
| | | Treatment | Treatment | Treatment |
| Age | Treatment | [%] | [%] | [%] |
| 30 yr | Placebo | 50.2 (0, 80) | 70.6 (50, 89) | 70.7 (53, 86) |
| | 30 mg | 77.7 (56, 92) | 77.6 (62, 91) | 76.8 (63, 89) |
| 50 yr | Placebo | 60.6 (0, 83) | 75.8 (56, 92) | 76.4 (62, 91) |
| | 30 mg | 82.4 (65, 94) | 82.6 (70, 93) | 81.1 (68, 93) |
| 65 yr | Placebo | 70.6 (40, 88) | 80.6 (64, 93) | 80.1 (65, 92) |
| | 30 mg | 86.0 (73, 95) | 86.0 (74, 96) | 84.3 (72, 95) |
| 75 yr | Placebo | 76.3 (50, 89) | 83.2 (68, 94) | 83.2 (71, 95) |
| | 30 mg | 88.1 (76, 96) | 88.0 (78, 96) | 86.5 (74, 96) |

Table 4 as shown below demonstrates that the proportion of patients who responded to Alitretinoin (PGA score of 1 or 2 at end of treatment) that did not relapse (e.g., those who did not have a PGA score of 5) 6 months post treatment was similar whether the duration of treatment was between 8 weeks and 24 weeks.

TABLE 4

| | | | Proportion of |
|---|---|---|---|
| | | Proportion | Responders Who |
| | | of Responders | Did Not Relapse |
| | | (PGA = 1 or 2) | (PGA ≠ 5) |
| | | at End of | 6 Months |
| Duration of | | Treatment | Post Treatment |
| Treatment | Treatment | [%] | [%] |
| 4 weeks | Placebo | 3.4 (1, 7) | 73.1 (54, 90) |
| | 30 mg | 4.3 (1, 8) | 79.2 (68, 89) |
| 8 weeks | Placebo | 10.2 (6, 15) | 74.7 (56, 92) |
| | 30 mg | 15.2 (10, 21) | 80.7 (70, 90) |
| 12 weeks | Placebo | 14.1 (9, 20) | 75.9 (56, 92) |
| | 30 mg | 23.7 (16, 31) | 82.3 (68, 95) |
| 16 weeks | Placebo | 15.4 (9, 21) | 77.0 (57, 92) |
| | 30 mg | 30.2 (23, 38) | 83.3 (72, 94) |
| 20 weeks | Placebo | 15.7 (10, 22) | 77.8 (59, 93) |
| | 30 mg | 35.4 (28, 43) | 83.9 (72, 94) |
| 24 weeks | Placebo | 16.1 (10, 23) | 78.5 (61, 93) |
| | 30 mg | 40.4 (33, 48) | 84.4 (75, 93) |

Table 5 demonstrates in greater detail the proportion of subjects with a PGA score of 1, 2 or 3 (evaluated individually) at end of treatment that did not relapse (e.g., those who did not have a PGA score of 5) 6 months post treatment was similar whether the duration of treatment was 8 weeks or 24 weeks.

TABLE 5

| | | Proportion | Proportion | Proportion |
|---|---|---|---|---|
| | | of Subjects | of Subjects | of Subjects |
| | | (PGA = 1) | (PGA = 2) | (PGA = 3) |
| Duration | | at End of | at End of | at End of |
| of | | Treatment | Treatment | Treatment |
| Treatment | Treatment | [%] | [%] | [%] |
| 4 weeks | Placebo | 1.3 (1, 2) | 2.9 (1, 6) | 9.1 (4, 14) |
| | 30 mg | 1.3 (1, 2) | 3.7 (1, 7) | 10.5 (5, 16) |
| 8 weeks | Placebo | 2.3 (1, 5) | 8.2 (4, 13) | 18.6 (13, 25) |
| | 30 mg | 3.6 (1, 7) | 11.7 (7, 17) | 22.5 (16, 29) |
| 12 weeks | Placebo | 3.2 (1, 6) | 10.9 (6, 16) | 21.6 (15, 29) |
| | 30 mg | 6.6 (3, 11) | 17.1 (11, 24) | 26.4 (19, 34) |
| 16 weeks | Placebo | 3.6 (1, 7) | 11.9 (7, 18) | 22.6 (16, 30) |
| | 30 mg | 9.2 (5, 14) | 21.0 (14, 28) | 27.7 (21, 35) |
| 20 weeks | Placebo | 3.8 (1, 7) | 11.9 (7, 17) | 23.2 (16, 30) |
| | 30 mg | 11.9 (7, 17) | 23.6 (17, 31) | 28.0 (21, 35) |
| 24 weeks | Placebo | 3.9 (1, 7) | 12.3 (7, 18) | 23.1 (16, 30) |
| | 30 mg | 14.7 (9, 21) | 25.7 (19, 33) | 28.2 (21, 35) |

Table 6 demonstrates in greater detail the proportion of subjects by PGA score of 1, 2 or 3 (evaluated individually) at end of treatment that did not relapse (e.g., those who did not have a PGA score of 5) 6 months post treatment was similar whether the duration of treatment was 8 weeks or 24 weeks.

TABLE 6

| | | | | Proportion of |
|---|---|---|---|---|
| | | Proportion of | Proportion of | Subjects with |
| | | Subjects with | Subjects with | PGA = 3 |
| | | PGA = 1 | PGA = 2 | Who Did |
| | | Who Did | Who Did | Not Relapse |
| | | Not Relapse | Not Relapse | (PGA ≠ 5) |
| Duration | | (PGA ≠ 5) | (PGA ≠ 5) | 6 Months Post |
| of | | 6 Months Post | 6 Months Post | Treatment |
| Treatment | Treatment | Treatment [%] | Treatment [%] | [%] |
| 4 weeks | Placebo | 60.0 (0, 83) | 72.0 (50, 91) | 70.7 (55, 85) |
| | 30 mg | 79.8 (61, 93) | 77.9 (64, 91) | 76.4 (62, 88) |
| 8 weeks | Placebo | 60.5 (0, 86) | 73.6 (54, 92) | 72.6 (57, 88) |
| | 30 mg | 81.0 (63, 94) | 79.5 (67, 92) | 77.8 (65, 89) |
| 12 weeks | Placebo | 61.6 (0, 86) | 74.7 (54, 92) | 74.1 (59, 89) |
| | 30 mg | 81.9 (64, 94) | 80.8 (68, 92) | 79.1 (67, 90) |
| 16 weeks | Placebo | 62.3 (0, 83) | 75.4 (55, 92) | 75.4 (61, 90) |
| | 30 mg | 82.5 (66, 94) | 81.7 (69, 92) | 80.1 (67, 91) |
| 20 weeks | Placebo | 63.1 (0, 86) | 76.0 (55, 92) | 76.3 (61, 90) |
| | 30 mg | 83.1 (67, 94) | 82.6 (69, 94) | 80.9 (68, 92) |
| 24 weeks | Placebo | 63.4 (4, 86) | 76.9 (58, 92) | 77.2 (62, 90) |
| | 30 mg | 83.7 (67, 94) | 83.3 (70, 95) | 81.6 (68, 92) |

In reviewing the data to make these determinations, all available PGA scores observed (not including scores derived from missing data such as last observation carried forward (LOCF) including those at baseline, every 4 weeks during treatment, and every 4 to 8 weeks after cessation of treatment for up to 72 weeks post treatment from the various clinical trials were used to develop a longitudinal dose response model.

Clinical trial simulations, using the longitudinal dose response model, were conducted and evaluated the following:
1) Magnitude of efficacy in PGA score at end of treatment (as the probability of a PGA score of 1 or 2, indicating “clear” or “almost clear”, respectively)
2) Time from the initial dose (placebo or Alitretinoin) to onset of effect (where onset is defined as the time to a 30% probability of at least a 2 point reduction in PGA score)
3) Proportion of responders at the end of treatment who did not relapse (i.e., had a PGA score ≠5) by 6-months post-treatment.

In clinical studies, efficacy was measured using PGA score and modified total lesion signs and symptoms (mTLSS) at various times during and post treatment. PGA scores over time were chosen for this analysis because PGA scores were the primary endpoint in the Phase III studies and were collected at more time points than the mTLSS. The PGA was graded according to 5 point scale (clear, almost clear, mild disease, moderate disease, severe disease). The mTLSS severity score was graded 0 to 3 for 7 parameters.

As used herein a “Time to onset of effect” is the time from the initial dose (alitretinoin or placebo) to a 2 point reduction in the PGA score in at least 30% of subjects.

The patient population in which this analysis is based has been found to have alitretinoin demonstrating some efficacy in at least one third of the subjects; and >99% of subjects have a baseline score of 5, so a two point reduction represents a substantial improvement in disease status. A >2 weeks time to onset is considered clinically relevant.

For the Physician Global Assessment (PGA), a general description of CHE severity, which is a well recognized scale is shown below in Table 7 for clarification purposes.

TABLE 7

| PGA Severity | Features | Intensity | Area Involved^{a} |
|---|---|---|---|
| Severe | Erythema, Scaling, | At least | >30% of |
| | Hyperkeratosis/ | one moderate | affected |
| | Lichenification | or severe | hand surface |
| | Vesiculation, | At least | |
| | Edema, Fissures, | one severe | |
| | Pruritus/Pain | | |
| Moderate | Erythema, Scaling, | At least | 10%-30% of |
| | Hyperkeratosis/ | one mild | affected |
| | Lichenification | or moderate | hand surface |
| | Vesiculation, | At least | |
| | Edema, Fissures, | one moderate | |
| | Pruritus/Pain | | |
| Mild | Erythema, Scaling, | At least | Less than |
| | Hyperkeratosis/ | one mild | 10% of |
| | Lichenification | | affected |
| | Vesiculation, | At least | hand |
| | Edema, Fissures, | one mild | surface |
| | Pruritus/Pain | | |
| Almost Clear | Erythema, Scaling, | At least | Less than |
| | Hyperkeratosis/ | one mild | 10% of |
| | Lichenification | | affected |
| | Vesiculation, | Absent | hand |
| | Edema, Fissures, | surface | |
| | Pruritus/Pain | | |
| Clear | Erythema, Scaling, | Absent | Not detectable |
| | Hyperkeratosis/ | | |
| | Lichenification | | |
| | Vesiculation, | Absent | |
| | Edema, Fissures, | | |
| | Pruritus/Pain | | |

^{a}Area involved does not apply to patients with fingertip eczema. In this assessment, affected hand surface refers to the surface area of the more severely affected side (palm or dorsum) of the more affected hand.

The above description fully discloses the invention including preferred embodiments thereof Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. A method of improving the efficacy of Alitretinoin for the treatment of Severe Refractory Chronic Hand Eczema (CHE) in a patient in need thereof comprising administering to said patient an effective amount of Alitretinoin and wherein the age of the patient is 50 years old or greater.

2. The method according to claim 1 wherein the amount of Alitretinoin is from about 10 mg to about 40 mg per day.

3. The method according to claim 2 wherein the amount of Alitretinoin is 30 mg.

4. The method according to claim 1 wherein the Alitretinoin is administered for up to 24 weeks.

5. The method according to claim 4 wherein the Alitretinoin is administered for up to 20 weeks, or up to 16 weeks

6. The method according to claim 4 wherein the Alitretinoin is administered for up to 12 weeks.

7. The method according to claim 4 wherein the Alitretinoin is administered for up to 8 weeks.

8. The method according to claim 1 wherein the disease severity of the CHE is evaluated using a modified total lesion symptom score (mTLSS) and/or a PGA scale.

9. The method according to claim 8 wherein the patient achieves a PGA score of 1 or 2 during the course of treatment with Alitretinoin.

10. The method according to claim 9 wherein the course of treatment is discontinued upon reaching a PGA score or 1 or 2.

11. The method according to claim 9 wherein the course of treatment is less than a 24 weeks duration of treatment.

12. The method of treatment according to claim 1 wherein the patient is 60 years old or greater.

13. The method of treatment according to claim 1 wherein the patient is 65 years old or greater.

14. The method of treatment according to claim 1 wherein the patient is 70 years old or greater, or the patient is 75 years old or greater.

15. The method of treatment according to claim 1 wherein the patient population of 50 years or greater experiences a faster onset of action, and/or a higher response rate to the treatment with Alitretinoin, and/or a greater chance of not relapsing 6 months after treatment.

16. The method of treatment according to claim 1 wherein the time to relapse post treatment is 6 months or greater.

17. The method of treatment according to claim 16 wherein the time to relapse post treatment is 9 months or greater.

18. The method according to according to claim 1 wherein whether or not a patient relapses is not influenced by the duration of treatment.

19. A method of obtaining optimal dosing of Alitretinoin for the treatment of Severe Refractory Chronic Hand Eczema (CHE) in a patient in need thereof comprising administering to said patient an effective amount of Alitretinoin, the age of the patient is 50 years old or greater, and the patient is treated until a PGA score of clear or almost clear is reached.

20. The method of treatment according to claim 19 wherein the patient is 60 years old or greater; or the patient is 65 years old or greater.

21. The method of treatment according to claim 19 wherein the patient is 70 years old or greater, or the patient is 75 years old or greater.

22. The method of treatment according to claim 19 wherein the time to relapse post treatment is 6 months or greater.

23. The method of treatment according to claim 22 wherein the time to relapse post treatment is 9 months or greater.

24. The method of treatment according to claim 19 wherein the course of treatment is 20 weeks or less in duration of treatment, or is 16 weeks or less in duration of treatment.

25. The method according to claim 19 wherein the amount of Alitretinoin administered daily is from 10 mg to 40 mg.

26. The method according to claim 25 wherein the amount of Alitretinoin administered daily is 10 mg or 30 mg.

27. The method of treatment according to claim 19 wherein there are less patients that relapse 6 months post-treatment in the 50 year of age or greater population as compared to placebo or compared to a population of less than 50 years of age.

28. The method according to claim 19 wherein the time to relapse is 6 months or greater in >83% of subjects.

29. The method of treatment according to claim 19 wherein the treatment time to a PGA score of clear or almost clear is less than the duration of treatment as compared to placebo or to a population less than 50 years of age.

30. The method of treatment according to claim 19 wherein the patient population of 50 years or greater is less likely to incur an increased risk from treatment with Alitretinoin as compared to placebo or to a population less than 50 years of age.

31. The method of treatment according to claim 19 wherein the elderly patient population experiences a faster onset of action and/or a higher response rate to the treatment with Alitretinoin.

32. The method of treatment according to claim 19 wherein the time to relapse is not influenced by the duration of treatment.
